(19) United States
(12) Patent Application Publication
(10) Pub. No.: US 2026/0345281 A1
(43) Pub. Date: Nov. 5, 2026

(54) DETERMINING PLACEMENT QUALITY OF A COMPONENT BASED ON MOTION OF A REMOTE CENTER OF MOTION DURING A STRAIN RELIEF OPERATION

(71) Applicant: INTUITIVE SURGICAL OPERATIONS, INC., Sunnyvale, CA (US)

(72) Inventor: Pavel CHTCHEPROV, Durham, NC (US)

(21) Appl. No.: 19/486,325

(22) PCT Filed: Jun. 13, 2024
(86) PCT No.: PCT/US2024/033824

§ 371 Date: Nov. 20, 2025

Related U.S. Application Data
(60) Provisional application No. 63/508,194, filed on Jun. 14, 2023.

(51) Int. Cl. A61B 90/00; A61B 34/20; A61B 34/30; A61B 34/35

(52) U.S. Cl. CPC A61B 90/03; A61B 34/20; A61B 34/35; A61B 2034/302; A61B 2090/031; A61B 2090/062; A61B 2090/067

(57) ABSTRACT

A computer-assisted system includes a repositionable structure configured to pivot a component about a remote center of motion (RCM) during operation, where the repositionable structure comprises a plurality of joints. The computer-assisted system further includes a processor system communicatively coupled to the repositionable structure, where the processor system is configured to: command the repositionable structure to perform a strain relief operation by floating one or more joints of the plurality of joints, wherein floating the one or more joints facilitates external manipulation of the repositionable structure that moves the RCM, determine a motion of the RCM occurring during the strain relief operation, and determine a placement quality of the component based on the motion of the RCM.

### RELATED APPLICATIONS

This application claims the benefit to U.S. Provisional Application No. 63/508,194, filed Jun. 14, 2023, and entitled “Determining Placement Quality of a Component Based on Motion of a Remote Center of Motion During a Strain Relief Operation,” the subject matter of which is incorporated by reference herein.

### TECHNICAL FIELD

The present disclosure relates generally to computer-assisted systems and more particularly to determining placement quality of a component based on motion of a remote center of motion during a strain relief operation.

### BACKGROUND

Some computer-assisted systems are configured to support one or more instruments that are moved, energized, or articulated to perform various procedures. The computer-assisted system can be automated, semi-automated, teleoperated, etc. The computer-assisted system can include a repositionable structure having a remote center of motion (RCM) about which a part of the repositionable structure, or a component supported by the repositionable structure, pivots during movement. In a medical example, the RCM can be located at a point coinciding with a cannula surface or cannula interior space, that experiences little to no translation during a procedure performed with the repositionable structure. In some medical examples, an instrument mounted to a repositionable structure of a computer-assisted system is inserted through a cannula into a workspace, and is used to perform tasks in the workspace. The computer-assisted system moves the repositionable structure to pivot part of the repositionable structure, along with the instrument mounted on the repositionable structure, about the RCM. In some examples, the computer-assisted system may have multiple repositionable structures, each having a respective RCM, and each configured to support one or more instruments.

In a medical example, the location of the RCM is relative to a location where the instrument is inserted into the workspace (e.g., a patient body wall) and may become unsatisfactory throughout a teleoperated surgical procedure. Such unsatisfactory RCM positioning can cause undesirable strain to the patient body wall and can be difficult for an operator to detect.

Accordingly, improved techniques for determining a placement quality of a component in a computer-assisted system are desirable.

### SUMMARY

Consistent with some embodiments, a computer-assisted system includes a repositionable structure configured to pivot a component about a remote center of motion (RCM) during operation, where the repositionable structure comprises a plurality of joints. The computer-assisted system further includes a processor system communicatively coupled to the repositionable structure, where the processor system is configured to: command the repositionable structure to perform a strain relief operation by floating one or more joints of the plurality of joints, wherein floating the one or more joints facilitates external manipulation of the repositionable structure that moves the RCM, determine a motion of the RCM occurring during the strain relief operation, and determine a placement quality of the component based on the motion of the RCM.

Consistent with some embodiments, a method comprises: commanding, by a processor system, a repositionable structure to perform a strain relief operation by floating one or more joints of a plurality of joints of the repositionable structure, wherein floating the one or more joints facilitates external manipulation of a repositionable structure that moves a remote center of motion (RCM), where the repositionable structure is configured to pivot a component about the RCM during operation; determining, by the processor system, a motion of the RCM occurring during the strain relief operation; and determining, by the processor system, a placement quality of the component based on the motion of the RCM.

Consistent with some embodiments, one or more non-transitory machine-readable media include a plurality of machine-readable instructions which when executed by a processor system are adapted to cause the processor system to perform any of the methods described herein.

### BRIEF DESCRIPTION OF THE DRAWINGS

FIG. 1 is a simplified diagram of a computer-assisted system in accordance with one or more embodiments.

FIG. 2 is a simplified diagram showing a computer-assisted system in according to some embodiments.

FIG. 3 is a flow diagram illustrating a method for determining a placement quality of a component in accordance with one or more embodiments.

FIG. 4 is a flow diagram of method steps for performing a strain relief operation in a computer-assisted system in accordance with one or more embodiments.

FIGS. 5A-5F illustrate examples of how to determine how an RCM is positioned relative to a target range based on motion during a strain relief operation.

In the figures, elements having the same designations have the same or similar functions.

### DETAILED DESCRIPTION

In this description, specific details are set forth describing some embodiments consistent with the present disclosure. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional.

Further, the terminology in this description is not intended to limit the invention. For example, spatially relative terms-such as “beneath”, “below”, “lower”, “above”, “upper”, “proximal”, “distal”, and the like-may be used to describe the relation of one element or feature to another element or feature as illustrated in the figures. These spatially relative terms are intended to encompass different positions (i.e., locations) and orientations (i.e., rotational placements) of the elements or their operation in addition to the position and orientation shown in the figures. For example, if the content of one of the figures is turned over, elements described as “below” or “beneath” other elements or features would then be “above” or “over” the other elements or features. A device may be otherwise oriented and the spatially relative descriptors used herein interpreted accordingly. Likewise, descriptions of movement along and around various axes include various special element positions and orientations. In addition, the singular forms “a”, “an”, and “the” are intended to include the plural forms as well, unless the context indicates otherwise. And, the terms “comprises”, “comprising”, “includes”, and the like specify the presence of stated features, steps, operations, elements, and/or components but do not preclude the presence or addition of one or more other features, steps, operations, elements, components, and/or groups. Components described as coupled may be electrically or mechanically directly coupled, or they may be indirectly coupled via one or more intermediate components.

Elements described in detail with reference to one embodiment, implementation, or module may, whenever practical, be included in other embodiments, implementations, or modules in which they are not specifically shown or described. For example, if an element is described in detail with reference to one embodiment and is not described with reference to a second embodiment, the element may nevertheless be claimed as included in the second embodiment. Thus, to avoid unnecessary repetition in the following description, one or more elements shown and described in association with one embodiment, implementation, or application may be incorporated into other embodiments, implementations, or aspects unless specifically described otherwise, unless the one or more elements would make an embodiment or implementation non-functional, or unless two or more of the elements provide conflicting functions.

In some instances, well known methods, procedures, components, and circuits have not been described in detail so as not to unnecessarily obscure aspects of the embodiments.

This disclosure describes various devices, elements, and portions of computer-assisted systems and elements in terms of their state in three-dimensional space. As used herein, the term “position” refers to the location of an element or a portion of an element (e.g., three degrees of translational freedom in a three-dimensional space, such as along Cartesian x-, y-, and z-coordinates). As used herein, the term “orientation” refers to the rotational placement of an element or a portion of an element (e.g., three degrees of rotational freedom in three-dimensional space, such as about roll, pitch, and yaw axes, represented in angle-axis, rotation matrix, quaternion representation, and/or the like). As used herein, and for a device with a kinematic series, such as with a repositionable structure with a plurality of links coupled by one or more joints, the term “proximal” refers to a direction toward a base of the kinematic series, and “distal” refers to a direction away from the base along the kinematic series.

As used herein, the term “pose” refers to the multi-degree of freedom (DOF) spatial position and orientation of a coordinate system of interest attached to a rigid body. In general, a pose includes a pose variable for each of the DOFs in the pose. For example, a full 6-DOF pose for a rigid body in three-dimensional space would include 6 pose variables corresponding to the 3 positional DOFs (e.g., x, y, and z) and the 3 orientational DOFs (e.g., roll, pitch, and yaw). A 3-DOF position only pose would include only pose variables for the 3 positional DOFs. Similarly, a 3-DOF orientation only pose would include only pose variables for the 3 rotational DOFs. Further, a velocity of the pose captures the change in pose over time (e.g., a first derivative of the pose). For a full 6-DOF pose of a rigid body in three-dimensional space, the velocity would include 3 translational velocities and 3 rotational velocities. Poses with other numbers of DOFs would have a corresponding number of velocities translational and/or rotational velocities.

Aspects of this disclosure are described in reference to computer-assisted systems, which can include devices that are teleoperated, externally manipulated, autonomous, semiautonomous, and/or the like. Further, aspects of this disclosure are described in terms of an implementation using a teleoperated surgical system, such as the da Vinci® Surgical System commercialized by Intuitive Surgical, Inc. of Sunnyvale, California. Knowledgeable persons will understand, however, that inventive aspects disclosed herein may be embodied and implemented in various ways, including teleoperated and non-teleoperated, and medical and non-medical embodiments and implementations. Implementations on a da Vinci® Surgical Systems are merely exemplary and are not to be considered as limiting the scope of the inventive aspects disclosed herein. For example, techniques described with reference to surgical instruments and surgical methods may be used in other contexts. Thus, the instruments, systems, and methods described herein may be used for humans, animals, portions of human or animal anatomy, industrial systems, general robotic, or teleoperated systems. As further examples, the instruments, systems, and methods described herein may be used for non-medical purposes including industrial uses, general robotic uses, sensing or manipulating non-tissue work pieces, cosmetic improvements, imaging of human or animal anatomy, gathering data from human or animal anatomy, setting up or taking down systems, training medical or non-medical personnel, and/or the like. Additional example applications include use for procedures on tissue removed from human or animal anatomies (with or without return to a human or animal anatomy) and for procedures on human or animal cadavers. Further, these techniques can also be used for medical treatment or diagnosis procedures that include, or do not include, surgical aspects.

The disclosed techniques aid in determining placement quality of an RCM of a computer-assisted system relative to a location where an instrument is inserted through an opening and into the workspace. The location of the RCM may have a correlated translational relationship relative to a manipulator of the computer-assisted system, to a location of a body opening of a patient where an instrument is inserted into the patient, to a location of a cannula inserted into the patient at the body opening, and/or to a location of the instrument inserted into the patient at the body opening (e.g., without being inserted through a cannula). The RCM placement quality may be unsatisfactory if the RCM is positioned “too deeply” into the workspace (e.g., beyond a central plane of the opening into the workspace). Alternatively or additionally, the placement quality may be unsatisfactory if the RCM is positioned “too shallowly” into the workspace (e.g., not having reached a central plane of the opening into the workspace). Such unsatisfactory RCM positioning can lead to undesirable lateral motion of a component of the computer system, such as the correlated cannula or instrument, relative to the body opening during teleoperation. More generally, the component can be a portion of an instrument, the cannula, one or more joint(s), the RCM, and/or the like. Specifically, this can lead to the cannula or instrument exerting undesirable lateral forces on the patient body wall at an incision site during teleoperation. To mitigate the effects of these lateral forces, the disclosed techniques can detect, and aid in the correction of, unsatisfactory placement quality of the RCM.

The disclosed techniques aid in detecting RCM positioning that is outside of a target range (e.g., a target depth range) by observing and detecting the effect of lateral forces on an RCM-correlated cannula or an RCM-correlated instrument resulting from a strain relief operation. This can be used, for example, to help determine whether the RCM is positioned within a target depth range relative to a body wall. As an example, this strain relief operation may be performed by the system in response to the insertion of an instrument through a cannula. During the strain relief operation, the system temporarily floats one or more joints of a repositionable structure, such as by continuously updating the commanded positions of actuators driving relevant joints of a repositionable structure to their respective current positions and releasing brakes on one or more of the relevant joints for a brief period of time. The releasing allows lateral forces on the cannula or instrument, exerted by the patient body wall, to move the cannula or instrument toward an equilibrium position. If the RCM is positioned too deeply or too shallowly, the motion of the floated joint(s), the movement of the RCM-correlated cannula or RCM-correlated instrument, or the RCM may exceed a component distance threshold or an RCM distance threshold. The system can check for the relevant motion exceeding the relevant distance threshold and take appropriate action. For example, the system can enter a safe mode, disallow further movement of the repositionable structure associated with the motion exceeding the threshold, generate an alert that can be sensed by a human operator, provide a signal indicating the excess motion, etc. If the relevant motion does not exceed the relevant distance threshold, the system can be allowed to continue performing the operation, be teleoperated, etc. in response.

The position of the RCM relative to the patient (e.g., a patient opening) can change during the procedure, such as due to patient movement relative to the computer-assisted system. Such RCM motion relative to the patient may occur while the RCM location remains fixed to a world frame or to a base frame associated with the computer-assisted system. Therefore, even if the RCM is placed within the target range at the beginning of the procedure, the RCM can move out of position during the procedure, with or without knowledge of the computer-assisted system and/or operator. Further, the placement of the RCM outside of the target range may exist from before the procedure, which may occur when the computer-assisted system is set up by an inexperienced operator.

The disclosed techniques may automatically determine various indications regarding when the strain relief operation should be performed, perform the strain relief operation upon detecting one or more of these indications, determine RCM motion resulted from the strain relief operation, and/or alert the operator (or takes some other action) if the strain relief operation resulted in too much RCM motion. For simplicity, the following description refers to the cannula, but it is understood that the same results can be obtained using the instrument for scenarios where the instrument is directly inserted into the workspace through the patient opening without going through a cannula. In brief, at a first time, a prior strain relief operation resets the strain of lateral forces on a cannula by an opening to the workspace to zero or to within an acceptable range that is near zero. The position of the cannula (e.g., a distal cannula region) after the end of the prior strain relief operation acts as the starting position of the cannula, referred to as the first position. Subsequently, at a second time, teleoperated movement of the repositionable structure and associated components can cause the cannula (e.g., the distal cannula region) to move to a second position, while the RCM does not move (e.g., relative to the world frame or to the base frame associated with the computer-assisted system). The direction from the first position of the cannula to the second position of the cannula determines a first direction. At a third time, a strain relief operation is performed, causing the RCM to move from the position of the RCM before the strain relief operation to a position of the RCM after the strain relief operation. The direction of the RCM motion (e.g., a net motion) during the strain relief operation determines a second direction. The first direction and the second direction are compared to determine whether the RCM is placed too deeply or not deep enough. In scenarios where there has not been a prior strain relief operation, the first position for establishing the first direction may be determined based on the position of the cannula when the computer-assisted system enters a teleoperation mode. In general, the lateral forces on the cannula applied by the opening to the workspace are zero or are within an acceptable range that is near zero when the computer-assisted system enters teleoperation mode. In some examples, a control module determines the positions of the cannula and the RCM via forward kinematic information of the repositionable structure, which allows the control module to track the position of the cannula and the RCM relative to the world frame or to the base frame associated with the computer-assisted system. As a result, the control module can track the motion of the cannula during teleoperation (e.g., to determine the first direction) and the motion of the RCM during the strain relief operation (e.g., to determine the second direction) relative to the world frame or to the base frame associated with the computer-assisted system. In some examples, upon receiving an alert that the strain relief operation resulted in too much RCM motion, guidance may be provided such that operator may adjust the cannula such that the RCM is moved within a target depth range.

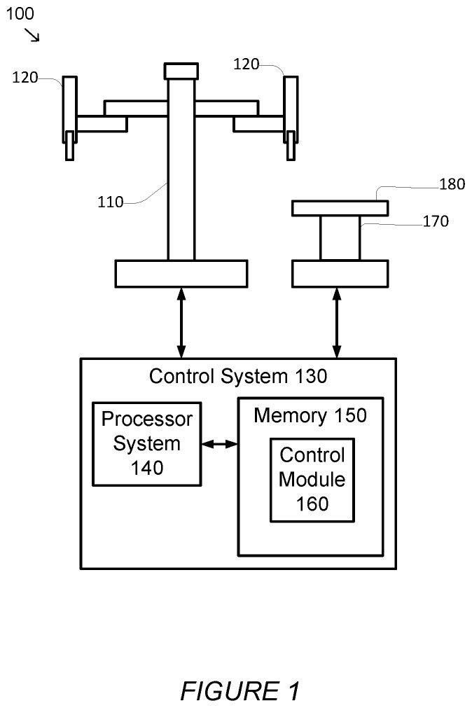

FIG. 1 is a simplified diagram of an example computer-assisted system 100, according to various embodiments. As shown in FIG. 1, computer-assisted system 100 includes a device 110 with one or more movable or repositionable structures 120. Each of the one or more repositionable structures 120 supports one or more end effectors. In some examples, device 110 may be consistent with a computer-assisted surgical device. The one or more repositionable structures 120 each provides support for one or more instruments, surgical instruments, imaging devices, and/or the like mounted to a distal end of at least one of the repositionable structures 120. Device 110 may further be coupled to an operator workstation (not shown), which may include one or more input devices for operating the device 110, the one or more repositionable structures 120, and/or the end effectors. In some embodiments, device 110 and the operator workstation may correspond to a da Vinci® Surgical System commercialized by Intuitive Surgical, Inc. of Sunnyvale, Calif. In some embodiments, computer-assisted surgical devices with other configurations, fewer or more repositionable structures 120, and/or the like may optionally be used with computer-assisted system 100.

Device 110 is coupled to a control system 130 via an interface. The interface may include one or more wireless links, cables, connectors, and/or buses and may further include one or more networks with one or more network switching and/or routing devices. Control system 130 includes a processor system 140 coupled to memory 150. Operation of control system 130 is controlled by processor system 140. And although control system 130 is shown with only one processor system 140, it is understood that processor system 140 may be representative of one or more central processing units, multi-core processors, microprocessors, microcontrollers, digital signal processors, field programmable gate arrays (FPGAs), application specific integrated circuits (ASICs), and/or the like in control system 130. Control system 130 may be implemented as a stand-alone subsystem and/or board added to a computing device or as a virtual machine. In some embodiments, control unit may be included as part of the operator workstation and/or operated separately from, but in coordination with the operator workstation.

Memory 150 is used to store software executed by control system 130 and/or one or more data structures used during operation of control system 130. Memory 150 may include one or more types of machine-readable media. Some common forms of machine-readable media may include floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, and/or any other medium from which a processor or computer is adapted to read.

As shown, memory 150 includes a control module 160 that supports autonomous and/or semiautonomous control of device 110. Control module 160 may include one or more application programming interfaces (APIs) for receiving position, motion, and/or other sensor information from device 110, exchanging position, motion, and/or collision avoidance information with other control units regarding other devices, such as a surgical table and/or imaging device, and/or planning and/or assisting in the planning of motion for device 110, repositionable structures 120, and/or the end effectors of device 110. And although control module 160 is depicted as a software application, control module 160 may be implemented using hardware, software, and/or a combination of hardware and software.

In some embodiments, computer-assisted system 100 may be found in an operating room and/or an interventional suite. And although computer-assisted system 100 includes only one device 110 with two repositionable structures 120, one of ordinary skill would understand that computer-assisted system 100 may include any number of devices with repositionable structures 120 and/or end effectors of similar and/or different design from device 110. In some examples, each of the devices may include fewer or more repositionable structures 120 and/or end effectors.

Computer-assisted system 100 further includes a surgical table 170. Like the one or more repositionable structures 120, surgical table 170 supports articulated movement of a tabletop 180 relative to a base of surgical table 170. In some examples, the articulated movement of tabletop 180 may include support for changing a height, a tilt, a slide, a Trendelenburg orientation, and/or the like of tabletop 180. Although not shown, surgical table 170 may include one or more control inputs, such as a surgical table command unit for controlling the position and/or orientation of tabletop 180. In some embodiments, surgical table 170 may correspond to one or more of the surgical tables commercialized by Trumpf Medical Systems GmbH of Germany.

Surgical table 170 is also coupled to control system 130 via a corresponding interface. The interface may include one or more wireless links, cables, connectors, and/or buses and may further include one or more networks with one or more network switching and/or routing devices. In some embodiments, surgical table 170 may be coupled to a different control unit than control system 130. In some examples, control module 160 may include one or more application programming interfaces (APIs) for receiving position, motion, and/or other sensor information associated with surgical table 170 and/or tabletop 180. In some examples, control module 160 may plan and/or assist in the planning of motion for surgical table 170 and/or tabletop 180.

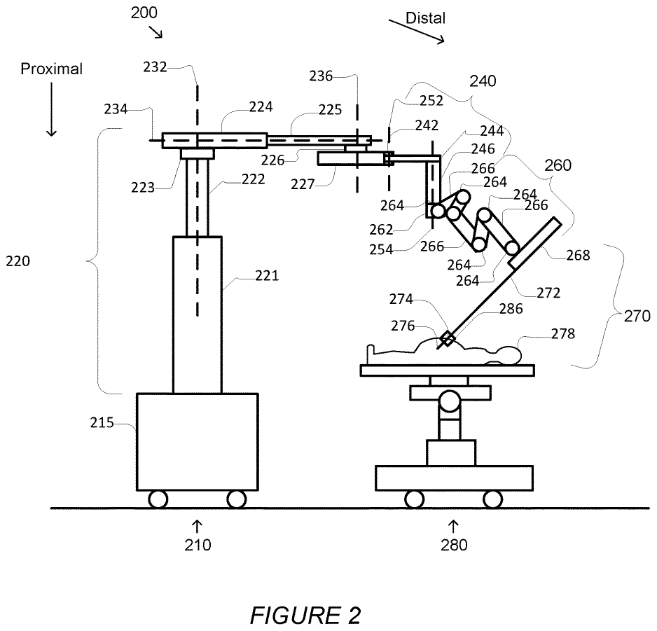

FIG. 2 is a simplified diagram showing a computer-assisted system 200 according to some embodiments. For example, the computer-assisted system 200 may be consistent with computer-assisted system 100. As shown in FIG. 2, the computer-assisted system 200 includes a computer-assisted device 210 with one or more repositionable structures and a surgical table 280. Although not shown in FIG. 2, the computer-assisted device 210 and the surgical table 280 are coupled together using one or more interfaces and one or more control units so that at least kinematic information about the surgical table 280 is known to the control module 160 being used to perform motion of the repositionable structures of the computer-assisted device 210.

The computer-assisted device 210 includes various links and joints. In the embodiments of FIG. 2, the computer-assisted device is generally divided into three different sets of links and joints. Starting at the proximal end with a mobile or patient-side mobile cart 215 is a set-up structure 220. Coupled to a distal end of the set-up structure is a series of links and set-up joints 240 forming a repositionable structure. And coupled to a distal end of the set-up joints 240 is a multi-jointed manipulator 260. In some examples, the series of set-up joints 240 and manipulator 260 may correspond to one of the repositionable structures 120. And although the computer-assisted device is shown with only one series of set-up joints 240 and a corresponding manipulator 260, one of ordinary skill would understand that the computer-assisted device may include more than one series of set-up joints 240 and corresponding manipulators 260 so that the computer-assisted device is equipped with multiple repositionable structures.

As shown, the computer-assisted device 210 is mounted on the mobile cart 215. The mobile cart 215 enables the computer-assisted device 210 to be transported from location to location, such as between operating rooms or within an operating room to better position the computer-assisted device in proximity to the tabletop 180. The set-up structure 220 is mounted on the mobile cart 215. As shown in FIG. 2, the set-up structure 220 includes a two-part column including column links 221 and 222. Coupled to the upper or distal end of the column link 222 is a shoulder joint 223. Coupled to the shoulder joint 223 is a two-part boom including boom links 224 and 225. At the distal end of the boom link 225 is a wrist joint 226, and coupled to the wrist joint 226 is an arm mounting platform 227.

The links and joints of the set-up structure 220 include various degrees of freedom for changing the position and orientation (i.e., the pose) of the arm mounting platform 227. For example, the two-part column is used to adjust a height of the arm mounting platform 227 by moving the shoulder joint 223 up and down along an axis 232. The arm mounting platform 227 is additionally rotated about the mobile cart 215, the two-part column, and the axis 232 using the shoulder joint 223. The horizontal position of the arm mounting platform 227 is adjusted along an axis 234 using the two-part boom. And the orientation of the arm mounting platform 227 may also adjusted by rotation about an arm mounting platform orientation axis 236 using the wrist joint 226. Thus, subject to the motion limits of the links and joints in the set-up structure 220, the position of the arm mounting platform 227 may be adjusted vertically above the mobile cart 215 using the two-part column. The positions of the arm mounting platform 227 may also be adjusted radially and angularly about the mobile cart 215 using the two-part boom and the shoulder joint 223, respectively. And the angular orientation of the arm mounting platform 227 may also be changed using the wrist joint 226.

The arm mounting platform 227 is used as a mounting point for one or more repositionable structures. The ability to adjust the height, horizontal position, and orientation of the arm mounting platform 227 about the mobile cart 215 provides a flexible set-up structure for positioning and orienting the one or more repositionable structures about a workspace located near the mobile cart 215 where an operation or procedure is to take place. For example, arm mounting platform 227 may be positioned above a patient so that the various repositionable structures and their corresponding manipulators and instruments have sufficient range of motion to perform a surgical procedure on the patient. FIG. 2 shows a single repositionable structure coupled to the arm mounting platform 227 using a first set-up joint 242. And although only one repositionable structure is shown, one of ordinary skill would understand that multiple repositionable structures may be coupled to the arm mounting platform 227 using additional first set-up joints.

The first set-up joint 242 forms the most proximal portion of the set-up joints 240 section of the repositionable structure. The set-up joints 240 may further include a series of joints and links. As shown in FIG. 2, the set-up joints 240 include at least links 244 and 246 coupled via one or more joints (not expressly shown). The joints and links of the set-up joints 240 include the ability to rotate the set-up joints 240 relative to the arm mounting platform 227 about an axis 252 using the first set-up joint 242, adjust a radial or horizontal distance between the first set-up joint 242 and the link 246, adjust a height of a manipulator mount 262 at the distal end of link 246 relative to the arm mounting platform 227 along an axis 254, and rotate the manipulator mount 262 about axis 254. In some examples, the set-up joints 240 may further include additional joints, links, and axes permitting additional degrees of freedom for altering a pose of the manipulator mount 262 relative to the arm mounting platform 227.

The manipulator 260 is coupled to the distal end of the set-up joints 240 via the manipulator mount 262. The manipulator 260 includes additional joints 264 and links 266 with an instrument carriage 268 mounted at the distal end of the manipulator 260. An instrument 270 is mounted to the instrument carriage 268. Instrument 270 includes a shaft 272, which is aligned along an insertion axis. The shaft 272 is typically aligned so that it passes through a remote center of motion (RCM) 274 associated with the manipulator 260 such that the shaft pivots about the RCM during teleoperation. In scenarios where a cannula is used and the instrument is inserted therethrough, the RCM coincides with a portion of the cannula such that the cannula pivots about the RCM during teleoperation. Location of the RCM 274 is typically maintained in a fixed translational relationship relative to the manipulator mount 262 so that operation of the joints 264 in the manipulator 260 result in rotations of the shaft 272 about the RCM 274. Depending upon the embodiment, the fixed translational relationship of the RCM 274 relative to the manipulator mount 262 is maintained using physical constraints in the joints 264 and links 266 of the manipulator 260, using software constraints placed on the motions permitted for the joints 264, and/or a combination of both. Representative embodiments of computer-assisted surgical devices using remote centers of motion maintained using physical constraints in joints and links are described in U.S. patent application Ser. No. 13/906,888 entitled “Redundant Axis and Degree of Freedom for Hardware-Constrained Remote Center Robotic Manipulator,” which was filed May 13, 2013, and representative embodiments of computer-assisted surgical devices using remote centers of motion maintained by software constraints are described in U.S. Pat. No. 8,004,229 entitled “Software Center and Highly Configurable Robotic Systems for Surgery and Other Uses,” which was filed May 19, 2005, the specifications of which are hereby incorporated by reference in their entirety. In some examples, the RCM 274 may correspond to a location of a body opening, such as an incision site or body orifice, in a patient 278 where shaft 272 is inserted into the patient 278. Because the RCM 274 corresponds to the body opening, as the instrument 270 is used, the RCM 274 remains stationary relative to the patient 278 to limit stresses on the anatomy of the patient 278 at the RCM 274. The RCM position relative to the patient may change as a surgical procedure commences despite the system's maintaining of the fixed translational relationship, such as due to patient movement relative to the robotic-assisted system. The disclosed techniques can detect such deviations and provide guidance to adjust the RCM position relative to the patient. In some examples, the shaft 272 may be optionally passed through a cannula (not shown) located at the body opening. In some examples, instruments having a relatively larger shaft or guide tube outer diameter (e.g., 4-5 mm or more) may be passed through the body opening using a cannula and the cannula may optionally be omitted for instruments having a relatively smaller shaft or guide tube outer diameter (e.g., 2-3 mm or less).

At the distal end of the shaft 272 is an end effector 276. The degrees of freedom in the manipulator 260 due to the joints 264 and the links 266 may permit at least control of the roll, pitch, and yaw of the shaft 272 and/or the end effector 276 relative to the manipulator mount 262. In some examples, the degrees of freedom in the manipulator 260 may further include the ability to advance and/or withdraw the shaft 272 using the instrument carriage 268 so that the end effector 276 may be advanced and/or withdrawn along the insertion axis and relative to the RCM 274. In some examples, the manipulator 260 may be consistent with manipulators for use with the da Vinci® Surgical System commercialized by Intuitive Surgical, Inc. of Sunnyvale, Calif. In some examples, the instrument 270 may be an imaging device such as an endoscope, a gripper, a surgical instrument such as a cautery or a scalpel, and/or the like. In some examples, the end effector 276 may include additional degrees of freedom, such as roll, pitch, yaw, grip, and/or the like that allow for additional localized manipulation of portions of the end effector 276 relative to the distal end of the shaft 272.

The control module 160 detects positioning of the RCM 274 outside of the target range by observing and detecting the effect of lateral forces on a cannula 286 resulting from a strain relief operation. The control module 160 uses this detection, for example, to help determine whether the RCM 274 “depth” relative to a body wall of the patient 278 is within the target range. This strain relief operation can be performed by the control module 160 in response to the insertion of an instrument 270 through the cannula 286 and/or through an opening into the workspace. During the strain relief operation, the control module 160 temporarily floats one or more joints of a repositionable structure, such as a repositionable structure 120 of FIG. 1, a multi-jointed manipulator 260 of FIG. 2, and/or the like. The control module floats the joints of the repositionable structure by continuously updating the commanded positions of actuators driving relevant joints of a repositionable structure to their respective current positions, releasing brakes on one or more of the relevant joints, and/or the like for a brief period of time. The releasing allows lateral forces on the cannula 286 to move the cannula 286 toward an equilibrium position. If the cannula 286 is inserted too deeply or too shallowly, then the motion of the floated joint(s), or the movement of the cannula 286, or the RCM 274, exceeds a component distance threshold or an RCM distance threshold, respectively. The control module 160 can check for the relevant motion exceeding the relevant distance threshold, and take appropriate action. For example, the control module can enter a safe mode, disallow further movement of the repositionable structure associated with the motion exceeding the distance threshold, generate an alert that can be sensed by a human operator, provide a signal indicating the excess motion, etc. If the relevant motion does not exceed the relevant distance threshold, then the control module 160 can allow the components of the computer-assisted system 100 and/or 200 to continue performing the operation, be teleoperated, etc. in response.

The position of the RCM 274 (e.g., indicative of the depth of the associated cannula 286) and/or the forces exerted externally on the cannula 286 can change during the procedure as the control module 160 manipulates the instruments 270. Therefore, even if the RCM 274 is placed within the target range at the beginning of the procedure, the RCM 274 can move out of position during the procedure, with or without knowledge of the operator. Further, the placement of the RCM 274 outside of the target range may exist from before the procedure. Even when the strain relief operation is performed before beginning the procedure, the initial placement may still be outside of the target range.

The control module 160 automatically determines various indications regarding when the strain relief operation should be performed. Upon detecting one or more of these indications, the control module 160 performs the strain relief operation and alerts the operator (or takes some other action) if the strain relief operation resulted in too much motion. Upon receiving the alert, the operator can adjust the cannula 286 to a better position, such as a more appropriate depth.

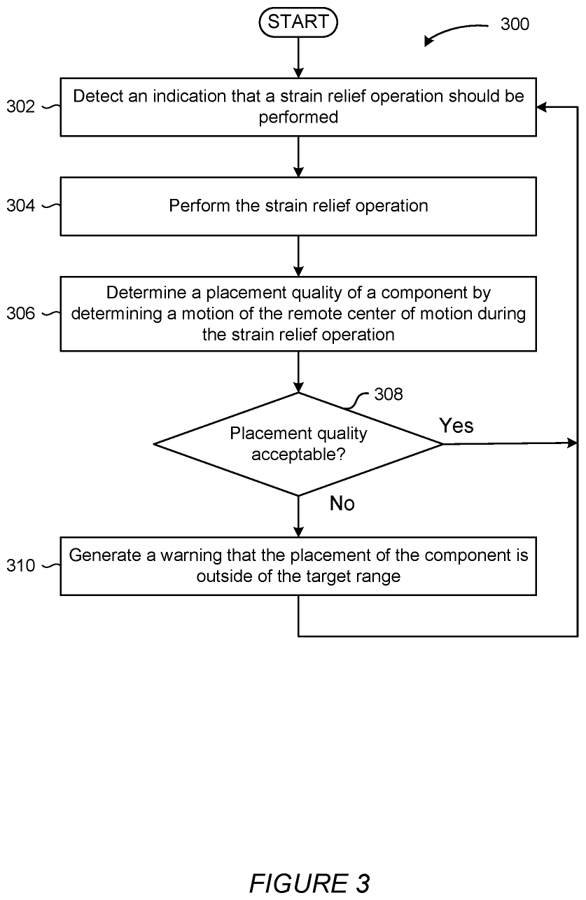

FIG. 3 is a flow diagram illustrating a method for determining a placement quality of a component in accordance with one or more embodiments. Although the method steps are described in conjunction with the system of FIGS. 1-2 and the examples of 5A-5F, persons of ordinary skill in the art will understand that any system configured to perform the method steps, in any order, is within the scope of the present disclosure. One or more of the processes 302-310 of method 300 can be implemented, at least in part, in the form of executable code stored on non-transient, tangible, machine-readable media. This executable code, when executed by a processor system (e.g., the processor system 140 in the control system 130), can cause the processor system to perform one or more of the processes 302-310. In some embodiments, method 300 can be performed by a module, such as the control module 160. In some embodiments, method 300 can be applied to one or more repositionable structures of a computer-assisted system to determine whether an RCM 274 of a computer-assisted system is positioned within a target range in accordance with one or more embodiments.

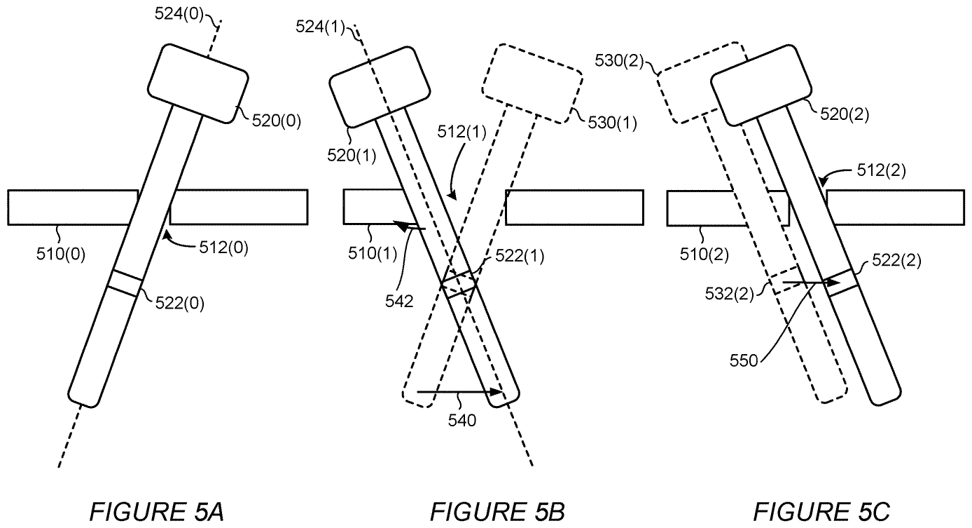

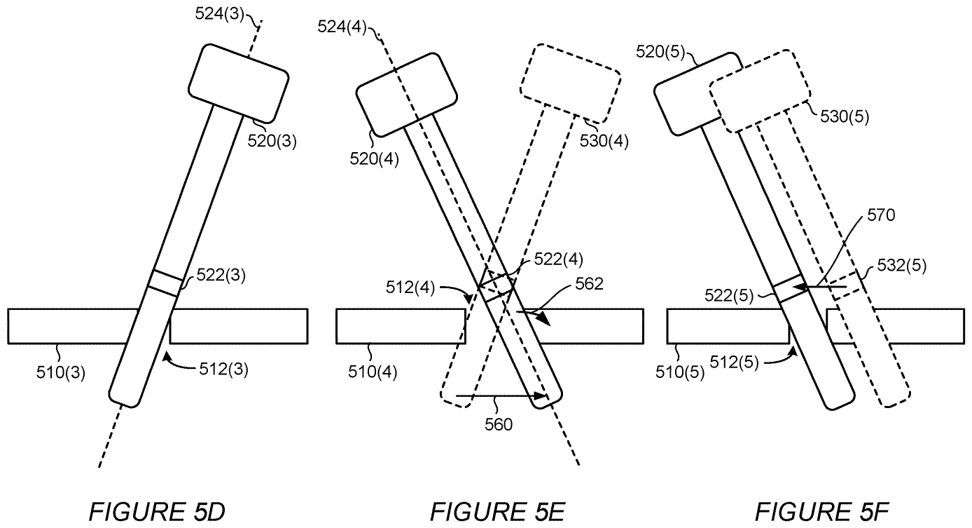

Aspects of method 300 are described via reference to FIGS. 5A-5F which illustrate examples of how to determine how an RCM 274 is positioned relative to a target range based on motion during a strain relief operation. However, it is understood that the examples of FIGS. 5A-5F are not restrictive, and that other repositionable structures, instruments, behaviors, and/or the like depicted in FIGS. 5A-5F may be different for computer-assisted systems 100 and/or 200, different repositionable structures, different imaging devices, different instruments, different DOFs, different procedures, and/or the like.

At a process 302, a control module, such as control module 160, detects an indication that a strain relief operation should be performed. The control module can employ various techniques, in any combination, to detect the indication that a strain relief operation should be performed. In some examples, the control module detects the indication that a strain relief operation should be performed by detecting the passage of a defined period of time since a previous strain relief operation was performed. In isolation, this technique provides for a periodic time out of a timer, after which a strain relief operation is performed. When used with one or more other techniques, this technique can be used to provide a maximum time limit between consecutive strain relief operations. In this latter case, if another technique generates an indication that a strain relief operation should be performed prior to the expiration of the current time out period, then the control module performs a strain relief operation prior to the end of the time out period. If no other technique generates an indication that a strain relief operation should be performed prior to the expiration of the current time out period, then the control module resets the timer and performs a strain relief operation at the expiration of the time out period. In either case, the control module resets the timer to begin a new timeout period.

In some examples, the control module detects the indication that a strain relief operation should be performed by detecting that the repositionable structure and/or one or more other components have stopped moving for a defined duration of time. In some examples, the control module detects the indication that a strain relief operation should be performed by detecting a command from the operator to perform a strain relief operation. The operator can generate the command by pressing one or more buttons on a component of the computer-assisted system 100 or 200, entering a command via a user interface, uttering a voice command, making a hand and/or body gesture, and/or the like. With this technique, the operator has explicit control as to when a strain relief operation is performed. For example, the operator can generate a command as each instrument 270 is installed, when a procedure begins, when an instrument 270 is removed and replaced with the same instrument 270 or a different instrument 270, at key points during the procedure, and/or the like.

In some examples, the control module detects the indication that a strain relief operation should be performed by detecting that an instrument 270 is installed on the relevant repositionable structure. With this technique, the control module automatically initiates a strain relief operation as each instrument 270 is installed prior to and/or during a procedure. This technique can be used as an automated way to perform strain relief operations during instrument 270 installation and insertion of instrument 270 through cannula 286. Additionally or alternatively, this technique can be used as a fallback technique if an operator forgets to manually generate a command to perform a strain relief operation when installing an instrument 270.

In some examples, the control module detects the indication that a strain relief operation should be performed by detecting that a distal portion of one or more instruments 270 supported by the repositionable structure is retracted back into the associated cannula(s) 286 or outside of the workspace, and/or by detecting a command for such retraction. When the distal portion of an instrument 270 is retracted back into the cannula 286 and/or is otherwise outside of the workspace, the strain relief operation can be performed in a condition where the instrument 270 is not able to interact, intentionally or unintentionally, with objects in the workspace. This technique can be used where unintended interaction of an instrument 270 with the workspace during a strain relief operation is undesirable.

In some examples, the control module detects the indication that a strain relief operation should be performed by determining that the instrument 270 supported by the repositionable structure is in view of an imaging device, such as a camera, an endoscope, and/or the like, and the instrument 270 is not in use. This technique reduces the likelihood of unintentional movement of an instrument 270 that is in use during a strain relief operation. Further, because the instrument 270 undergoing a strain relief operation is in view of the imaging device, the operator can view the movement of the instrument 270 and abort the strain relief operation if the instrument 270 appears to be moving in an undesirable manner. With this technique, the control module can determine that the computer-assisted system 100 or 200 is not in teleoperation mode. In this regard, some computer-assisted systems 100 and/or 200 exit teleoperation mode when in a camera control mode, an instrument clutch mode, a setup mode, and/or the like, when the instruments 270 are not being used to perform a procedure. Additionally or alternatively, the control module can determine that the computer-assisted system 100 or 200 is in teleoperation mode, but the computer-assisted system 100 or 200 is not performing any teleoperation commands. For example, the computer-assisted systems 100 and/or 200 can be programmed to be in a teleoperation mode, but teleoperated motion is not occurring. Teleoperated motion may not be occurring because the control module is not receiving commands (e.g., operator commands) from one or more input devices, or is not causing motion despite receiving commands from the one or more input devices. Additionally or alternatively, teleoperated motion may not be occurring because the control module is inhibiting teleoperated movement.

In some examples, the control module detects the indication that a strain relief operation should be performed by determining that a movement of an input device associated with the relevant repositionable structure and used to provide commands for desired motions of instrument 270 has exceeded an input device distance threshold from a reference position. If a movement of an input device has exceeded an input device distance threshold from the reference position, then the desired motions of the relevant repositionable structure and/or instrument 270 due to the commands from the input device may be exerting pressure on the cannula 286 and/or on the entrance to the workspace.

In some examples, the control module detects the indication that a strain relief operation should be performed by determining that a force greater than a force threshold is being exerted on the cannula 286. The force threshold can be determined based on the characteristics of the patient, or patient tissue, that would interact with the cannula 286. For example, where a cannula is inserted through an abdominal incision in an adult human, the force threshold may be set around 15, 17.5 20, 22.5, 25, 30, or other quantity of Newtons. Additionally or alternatively, the force threshold can be determined based on one or more of an operator preference, a type of instrument, a type of cannula, a type of procedure being performed, stage of procedure being performed, and/or the like. The control module can measure this force based on readings from force sensor(s) and/or pressure sensor(s) on the cannula 286, by force sensor(s), pressure sensor(s) and/or displacement sensor(s) on the repositionable structure, and/or the like. When a sensed force, pressure, and/or displacement has exceeded the force threshold, the relevant repositionable structure, the cannula 286, and/or instrument 270 may be exerting undesirable pressure on the entrance to the workspace (e.g., tissue of a patient). In some examples, the control module detects the indication that a strain relief operation should be performed by determining that a force greater than a force threshold (see example ranges above) or a torque greater than a torque threshold is being exerted on the cannula 286, such that a sufficiently large force and/or a sufficiently larger torque satisfies the condition(s) for performing a strain relief operation. For example, where a cannula is inserted through an abdominal incision in an adult human, the force threshold may be set at any of the values disclosed herein, and the torque threshold may be set around 1.5, 2.0, 2.5, 2.0, or other quantity of Newton-meters. Additionally or alternatively, the force threshold or the torque threshold can be set based on one or more of an operator preference, a type of instrument, a type of cannula, a type of procedure being performed, stage of the procedure being performed, and/or the like. The control module can measure the force or torque based on readings from force sensor(s), torque sensor(s), and/or pressure sensor(s) on the cannula 286, and/or determine the force or torque from joints sensor(s) of the repositionable structure, and/or the like. When a force has exceeded the force threshold or the torque has exceeded the torque threshold, the relevant repositionable structure, the cannula 286, and/or instrument 270 may be exerting undesirable pressure on the entrance to the workspace (e.g., tissue of a patient).

In some examples, at process 302, even if the control module detects an indication that a strain relief operation should be performed, the control module nevertheless does not perform a strain relief operation. In that regard, in some instances, the control module can refrain from performing a strain relief operation if the control module determines that an instrument 270 or a cannula 286 supported by the relevant repositionable structure has moved by more than a maximum distance. In some examples, the control module determines the positions of the instrument 270 and/or the cannula 286 via forward kinematics information of the repositionable structure, which allows the control module to track the position of the instrument 270 and/or the cannula 286 at all times. As a result, the control module can track the motion of the instrument 270 and/or the cannula 286 during teleoperation (e.g., to determine whether the instrument 270 and/or the cannula 286 have moved more than the maximum distance, thereby indicating that the control module should refrain from performing the strain relief operation). Additionally or alternatively, the control module can refrain from performing a strain relief operation when the control module determines that an instrument 270 or a cannula 286 supported by the relevant repositionable structure is at a depth that is greater than a maximum insertion depth. The maximum distance and/or the maximum insertion depth can be set based on one or more of an operator preference, a type of instrument, a type of cannula, a type of procedure being performed, and/or the like.

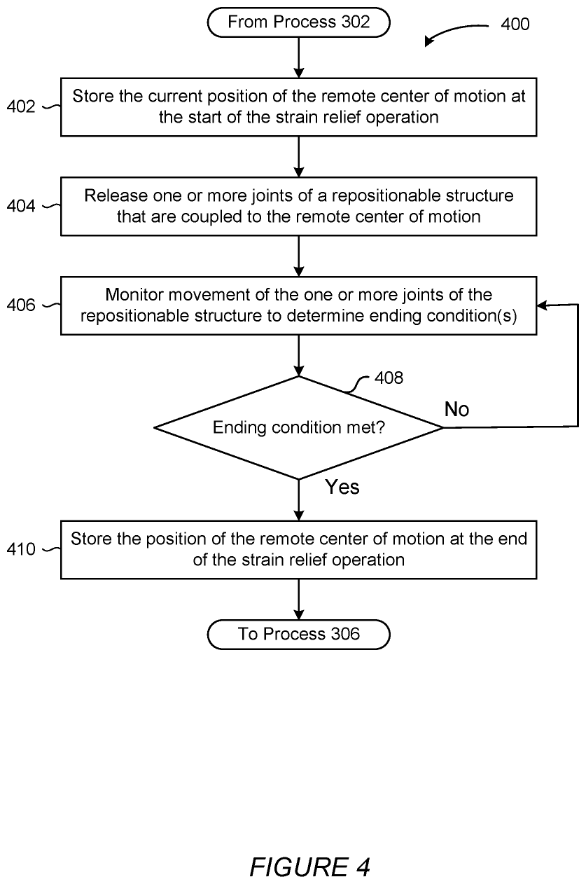

At a process 304, the control module performs the strain relief operation. The strain relief operation is described herein in conjunction with method 400 and illustrated in FIGS. 4 and 5A-5F. FIG. 4 is a flow diagram of method steps for performing a strain relief operation in a computer-assisted system in accordance with one or more embodiments. Although the method steps are described in conjunction with the system of FIGS. 1-2 and the examples of 5A-5F, persons of ordinary skill in the art will understand that any system configured to perform the method steps, in any order, is within the scope of the present disclosure. One or more of the processes 402-410 of method 400 can be implemented, at least in part, in the form of executable code stored on non-transient, tangible, machine-readable media. This executable code, when executed by a processor system (e.g., the processor system 140 in the control system 130), can cause the processor system to perform one or more of the processes 402-410. In some embodiments, method 300 can be performed by a module, such as the control module 160. In some embodiments, method 400 can be applied to one or more repositionable structures of a computer-assisted system to perform a strain relief operation in a computer-assisted system in accordance with one or more embodiments.

Aspects of method 400 are described via reference to FIGS. 5A-5F which illustrate examples of how to determine how an RCM is positioned relative to a target range based on motion during a strain relief operation. However, it is understood that the examples of FIGS. 5A-5F are not restrictive, and that other repositionable structures, cannulas, instruments, behaviors, and/or the like depicted in FIGS. 5A-5F may be different for computer-assisted systems 100 and/or 200, different repositionable structures, different imaging devices, different instruments, different DOFs, different procedures, and/or the like.

The method 400 begins at a process 402, where a control module, such as control module 160, stores the current position of the RCM 522 at the start of the strain relief operation. As shown in FIGS. 5A-5B, the current position of the RCM 522 indicates that a cannula 520 is placed too deeply into a workspace 510. At process 402, the control module stores the first position of the RCM 522(1) shown in FIG. 5B prior to performing the strain relief operation. Similarly, as shown in FIGS. 5D-5E, the current position of the RCM 522 indicates that a cannula 520 is placed too shallowly into a workspace 510. At process 402, the control module stores the first position of the RCM 522(4) shown in FIG. 5E prior to performing the strain relief operation. The control module can subsequently use the stored current position of the RCM 522 at the start of the strain relief operation to determine, at process 306 of method 300 as is discussed in further detail below, the motion of the RCM 522 during the strain relief operation.

At a process 404, the control module floats one or more joints of a repositionable structure that is coupled to the cannula 520. Floating one or more joints facilitates external manipulation of the repositionable structure by the forces exerted on the cannula that allows the RCM to move during the strain relief operation. These one or more joints include at least one joint whose motion can cause movement of the RCM 522 (and may include none, one, or more joints whose motion cannot cause movement of the RCM 522). As shown in FIG. 5C, when the control module floats one or more joints of the repositionable structure, the current position of the RCM 522 moves from a position of the RCM 532(2) at the start of the strain relief operation to a position of the RCM 522(2) at the end of the strain relief operation as indicated by movement direction 550. Similarly, as shown in FIG. 5F, when the control module floats one or more joints of the repositionable structure, the current position of the RCM 522 moves from a position of the RCM 532(5) at the start of the strain relief operation to a position of the RCM 522(5) at the end of the strain relief operation as indicated by movement direction 570.

The control module can float one or more joints of the repositionable structure. For example, a joint that is actuated is floated by continuously updating the commanded position of an actuator driving the actuated joint to its current position or updating the commanded velocity of the actuator driving the joint actuated joint to its current velocity, and a joint that is non-actuated is floated by releasing or partially releasing brakes on one or more of the relevant joints for a brief period of time. The releasing allows lateral forces on the cannula to move the cannula toward an equilibrium position. In some examples, the one or more joints include at least one joint that is not mechanically constrained from joint motion that moves the RCM. The one or more joints floated can include joints whose joint motion can move the RCM and/or joints whose joint motion cannot move the RCM. Further examples of techniques for the floating of joints are described in further detail in International Patent Application Publication No. WO 2015/142930, which is hereby incorporated by reference in its entirety.

Prior to the strain relief operation, the control module commands one or more actuators to move, resulting in movement of actuated joints to their current position. During the strain relief operation, the system temporarily floats one or more joints of a repositionable structure by continuously updating the commanded positions of actuators driving relevant joints of a repositionable structure to their respective current positions, and releasing brakes on one or more of the relevant joints for a brief period of time. The releasing allows lateral forces on the cannula to move the cannula toward an equilibrium position. If the cannula 520 is inserted too deeply, then lateral forces 542 on the cannula 520 cause the cannula 520 to move from a first position of the cannula 530(2) to a second position of the cannula 520(2). Correspondingly, the RCM 522 moves in movement direction 550 from a first position of the RCM 532(2) to a second position of the RCM 522(2). If the cannula 520 is inserted too shallowly, then lateral forces 562 on the cannula 520 cause the cannula 520 to move from a first position of the cannula 530(5) to a second position of the cannula 520(5). Correspondingly, the RCM 522 moves in movement direction 570 from a first position of the RCM 532(5) to a second position of the RCM 522(5). If the RCM is inserted too deeply or too shallowly and is outside of the target range, the motion of the floated joint(s), or the movement of the cannula or the RCM, may exceed a component distance threshold or an RCM distance threshold, respectively.

At a process 406, the control module monitors the movement of the one or more joints of a repositionable structure to determine an indication that a strain relief operation should end. The control module monitors the movement of the joints in order to determine such an indication that the current strain relief operation should end. The control module can employ various techniques, in any combination, to detect the indication that a strain relief operation should end.

In some examples, the control module detects the indication that a strain relief operation should end by determining that one or more components, or portions thereof, that are coupled to the computer-assisted system 100 and/or 200 have moved farther than a component distance threshold from a reference position, have moved faster than a component speed threshold, or have moved with undamped characteristics during the current strain relief operation. In some examples, the distance of the one or more components can be a portion of the distance in a particular direction rather than the overall distance travelled by the one or more components. Correspondingly, the component distance threshold can be a distance in the particular direction. Likewise, the speed of the one or more components can be a portion of the speed in a particular direction rather than the overall speed of the one or more components. Correspondingly, the component speed threshold can be a speed in the particular direction. In some examples, one or both of the component distance threshold and the component speed threshold can vary based on the direction of motion of the one or more components. The one or more portions can include a distal portion of the instrument 270 supported by the relevant repositionable structure. Additionally or alternatively, the one or more portions can include cannula 286. Additionally or alternatively, the one or more portions can include one or more joints or links of the relevant repositionable structure. Additionally or alternatively, the one or more portions can include the RCM 274 of the relevant repositionable structure.

The control module can set the reference position to the position of the relevant portion of the associated instrument 270, cannula 286, joint(s), or RCM 274 as of the last float of joint(s) of the repositionable structure. The last float of the joint(s) can be from a prior strain relief operation, a clutch operation, and/or the like. Representative embodiments for controlling a manipulator assembly including a manipulator arm and an instrument or tool by floating one or more joints and/or by controlling one or more floating joints in a clutch mode are described in U.S. Patent Application Publication No. 2022/0175472 entitled “Reducing Energy Buildup in Servo-Controlled Mechanisms,” which was filed Mar. 27, 2020. Representative embodiments for controlling a manipulator assembly in a clutch mode by driving one or more joints in response to an external articulation of another joint of the manipulator assembly are described in U.S. Patent Application Publication No. 2007/0013336 entitled “Software Center and Highly Configurable Robotic Systems for Surgery and Other Uses,” which was filed Jan. 18, 2007.

Additionally or alternatively, the control module can set the reference position to the position of the relevant portion of the associated instrument 270, cannula 286, joint(s), or RCM 274 immediately prior to the start of the current strain relief operation.

The control module can set the component distance threshold and/or the component speed threshold based on one or more of operator preference, a type of instrument 270, a type of cannula 286, and/or a type of procedure being performed. The component distance threshold can be in the range of 5 mm to 15 mm for certain procedures. The component speed threshold can be in the range of 1 mm/sec to 15 mm/sec for certain procedures. Additionally or alternatively, the control module can set the component distance threshold and/or the component speed threshold based on an activation status of the instrument 270 (e.g., if the instrument 270 is energized and delivering energy to a material such as tissue of a patient in a medical example). Additionally or alternatively, the control module can set the component distance threshold and/or the component speed threshold based on a kinematic configuration of the instrument 270. In one example, for an instrument that has operational jaws, the kinematic configuration can include an opening amount of the jaws, a clamping pressure of the jaws, a clamping force of the jaws, whether the jaws are grasping a material in the workspace, and/or the like. In various instances, the opening amount of the jaws can be determined as a binary manner (e.g., whether the jaws are open or closed), as a measurement of the amount the jaw is opened (e.g., an angle between the jaws, a linear or arcuate distance between tips of the jaws, etc.), or in some other manner (e.g., in multiple quantized amounts of opening such as fully open, half open, fully closed, etc.). In another example, for an instrument that has articulating joints, the kinematic configuration can include the amount of flexion or extension of the articulating joints, the pose of the links coupled to the articulating joints, and/or the like.

In some examples, the control module detects the indication that a strain relief operation should end by determining that a portion of an instrument 270 (e.g., a distal portion of the instrument 270) supported by the relevant repositionable structure, the cannula 286 supported by the relevant repositionable structure, the joint(s) floated in the repositionable structure, the link(s) of the repositionable structure, and/or the RCM 274 have stopped moving. In some examples, the control module detects the indication that a strain relief operation should end by determining that a portion of an instrument 270 supported by the relevant repositionable structure, the cannula 286 supported by the relevant repositionable structure, the joint(s) floated in the repositionable structure, the link(s) of the repositionable structure, and/or the RCM 274 are being moved in a disallowed direction.

In some examples, the control module detects the indication that a strain relief operation should end by determining that a duration of time has elapsed since the joint(s) of the repositionable structure were floated. For example, this duration of time can be in the range of 0.25 sec to 1.0 sec.

In some examples, the control module performs the strain relief operation as a series of abbreviated strain relief operations. This can be done by the control module ending and re-performing a set of abbreviated strain relief operations, each abbreviated strain relief operation lasting only a brief duration. With this approach, each abbreviated strain relief operation included in the series of abbreviated strain relief operations comprises a different portion of the strain relief operation. In this manner, the series of abbreviated strain relief operations has an effective duration of the entire strain relief operation, which in some instances can be the sum of the durations of time of the abbreviated strain relief operations included in the series of abbreviated strain relief operations. In some instances, such effective duration is equal to, or substantially equal to, the duration of the complete strain relief operation. Using this technique, the control module can spread out the motion of the component or portion thereof over a longer duration of time. By performing a series of abbreviated strain relief operations, the control module can limit the motion of the component or portion thereof during each abbreviated strain relief operation. While a series of abbreviated strain relief operations can result in an aggregate motion of the component that is greater than the motion of the component during a single non-abbreviated strain relief operation, the motion of the component during each abbreviated strain relief operation is limited.

At a process 408, the control module determines whether an indication that the strain relief operation should end is met. If no such indication is met, then the method 400 proceeds to process 406, described above, to continue to monitor the movement of the one or more joints of a repositionable structure.

Returning to process 408, if one or more such indications are met, then method 400 proceeds to process 410, where the control module stores the position of the RCM 274 at the end of the strain relief operation. As shown in FIG. 5C, as a result of the cannula 520 being placed too deeply into the workspace 510, the cannula 520 moves in movement direction 550 from a position of the RCM 532(2) at the start of the strain relief operation to a position of the RCM 522(2) at the end of the strain relief operation. The control module stores the position of the RCM 522(2) after the strain relief operation. Similarly, as shown in FIG. 5F, as a result of the cannula 520 being placed too shallowly into the workspace 510, the cannula 520 moves in movement direction 570 from a position of the RCM 532(5) at the start of the strain relief operation to a position of the RCM 522(5) at the end of the strain relief operation. The control module stores the position of the RCM 522(5) after the strain relief operation. The control module can subsequently use the stored position of the RCM 274 after the strain relief operation to determine, at process 306 of method 300 as is discussed in further detail below, the motion of the RCM 274 during the strain relief operation. After completing process 408, the strain relief operation is complete and control returns to process 306 of method 300.

Referring back to FIG. 3, at a process 306, the control module determines a placement quality of a component. In some embodiments, the component is an instrument 270, a cannula 286, a joint floated in the repositionable structure, a link of the repositionable structure, the RCM 274, and/or any plurality, subset, or portion thereof. As a first step in determining the placement quality of the component, the control module determines a motion of the RCM 274 during the strain relief operation. The control module determines the motion of the RCM 274 by comparing the position of the RCM 274 at the end of the strain relief operation, determined at process 410 of method 400, with the position of the RCM 274 at the start of the strain relief operation, determined at process 402 of method 400. Additionally or alternatively, the control module determines the motion of the RCM 274 by comparing the position of the RCM 274 at the end of the current strain relief operation, determined at process 410 of the current iteration of method 400, with the position of the RCM 274 at the end of the previous strain relief operation, determined at process 410 of the previous iteration of method 400.

The control module can then determine whether the placement quality of the component is acceptable, such as whether cannula 286 is too deeply or too shallowly placed based on comparing: (1) a first direction of motion of the distal portion of the cannula 286 and/or the distal portion of the instrument 270 from the end of the previous strain relief operation to the start of the current strain relief operation with (2) a second direction of motion of the RCM 274 during the current strain relief operation. For simplicity, the following description refers to the distal portion of the cannula 286, but it is understood that the same results can be obtained using the distal portion of the instrument 270, such as for scenarios where the instrument is directly inserted into the workspace through the patient opening without going through a cannula. In that regard, the first direction of motion can be a direction of a vector that begins at the position of the distal portion of the cannula 286 at the end of the previous strain relief operation and ends at the position of the distal portion of the cannula 286 at the start of the current strain relief operation. In some examples, the first direction can be a net motion of the cannula 286, the instrument 270, or other component or portion thereof, from a position at the end of a previous strain relief operation to a position at the start of the strain relief operation. This net motion represents the overall net motion of all teleoperation-induced deviations and/or motions. This net motion can result from a single teleoperated motion or from multiple teleoperated motions. In any case, the control module can determine the first direction directly from the net motion without separately determining the magnitudes and/or directions of the multiple teleoperated motions. The second direction of motion can be a direction of a vector that begins at the position of the RCM 274 at the start of the current strain relief operation and ends at the position of the RCM 274 at the end of the current strain relief operation. These vectors can represent the motion over the entire timeframe of the previous strain relief operation and/or the current strain relief operation. Additionally or alternatively, these vectors can represent the motion over any portion of the previous strain relief operation and/or the current strain relief operation. In various examples, the first direction and the second direction each represent a portion of the associated motion within a common plane or on a common axis.

In some examples, the first direction of motion can be determined via alternative techniques. Such techniques can be employed, for example, when the computer-assisted system 100 or 200 is initialized for a new procedure. When the computer-assisted system 100 or 200 is initialized, a strain relief operation has not yet been performed. Therefore, the first direction cannot be determined from a previous strain relief operation. In such cases, the control module determines the first direction of motion of the distal portion of the cannula 286 from when the computer-assisted system 100 or 200 enters teleoperation mode (but has not yet processed a teleoperative command) to the start of the current strain relief operation.

In some examples, the control module determines whether the component is too shallowly or too deeply inserted into the workspace based on the first direction and the second direction. In some examples, the first direction and the second direction can be confined to particular portions of the overall motion. For example, the first direction and the second direction can be determined without considering the motion in a direction perpendicular to the opening of the workspace, such as the direction into and out of the body wall. Instead, the first direction and/or the second direction can be determined based on motion of the RCM in a plane substantially parallel to the opening of the workspace. In some examples, the first direction and the second direction can correspond to cannula motion and RCM motion, respectively, along a common axis only. That axis can be determined based on an insertion axis corresponding to the instrument and/or cannula, an axis projected onto a plane parallel to the body wall, and/or the like.

In some examples, the control module determines an angular difference between the first direction and the second direction. In some embodiments, the control module determines the angular difference θ between the first direction and the second direction by setting a first unit vector \vec{D₁} that is aligned with the first direction and a second unit vector \vec{D₂} that is aligned with the second direction to have the same origin. The control module computes the dot product of the two vectors \vec{D₁}·\vec{D₂} which is equal to cos θ. The control module can then compute the angular difference in radians as θ=cos⁻¹(\vec{D₁}·\vec{D₂}). The angular difference can alternatively be expressed in degrees or other suitable angular unit of measure using different formulas.

Based on the direction of RC motion after the strain relief compared to the direction of the first and second proximal cannula alignments (portion of the cannula above the RCM), the control module determines whether the component is inserted too deeply into the workspace or too shallowly into the workspace. If the dot product of the vector defined by the motion of the RCM and the vector defined by \vec{D₂}−\vec{D₁} is greater than 0, indicating that the direction of the RCM motion is in the same direction as the proximal cannula motion, the RCM is inserted too shallowly into the workspace. On the other hand, if that dot product is less than 0, this indicates that the direction of the RCM is opposite that of the cannula motion and signifies that the RCM is inserted too deeply into the workspace.

The disclosed techniques are described with reference to motion of the distal portion of the cannula 286. However, in alternative embodiments, the disclosed techniques can be performed based on motion of any other suitable reference position, such as any other portion of the cannula that is not coincident with the RCM. As one example, the disclosed techniques can be performed based on motion of a proximal portion of the cannula. In such examples, the same computation as above applies, using the reference to determine the above's RCM motion vector.

As shown in FIG. 5A a cannula 520(0) is inserted through an opening 512(0) (e.g., a port, an incision point, a natural orifice, and/or the like) of a workspace 510(0) along an insertion axis 524(0). The pose of the cannula 520(0) reflects a pose after a prior joint float operation, such as a prior strain relief operation, a prior clutch operation, and/or the like. As further shown in FIG. 5A, cannula 520(0) is inserted too deeply into the workspace because a RCM 522(0) associated with cannula 520(0) is located within the interior of the workspace past an opening. As shown in FIG. 5B, the operator has generated commands to control an instrument (not shown) during a procedure. As a result of the commands, a repositionable structure (not shown) supporting cannula 520(1) has caused a change in the pose of cannula 520(1) from a first pose of the cannula 530(1) such that insertion axis 524(1) of the cannula 520(1) has rotated counterclockwise and the distal portion of the cannula 520(1) has moved in movement direction 540 to the right. As shown in FIG. 5C, the control module performs a strain relief operation. Lateral forces 542 on the cannula 520 cause the cannula 520 to move to the right from a first pose of the cannula 530(2) prior to the strain relief operation to a second pose of the cannula 520(2) after the strain relief operation. The lateral forces 542 on the left side of the opening 512(2) of the workspace 510(2) against the cannula 520 result in movement from the first pose of the cannula 530(2) to the second pose of the cannula 520(2). As a result, the RCM 522 likewise moves in movement direction 550 from a position of the RCM 532(2) at the start of the strain relief operation to a position of the RCM 522(2) at the end of the strain relief operation.

The control module determines that the movement direction 540 of the distal portion of the cannula 530(1) after the prior joint float operation to the pose of the distal portion of the cannula 520(1) prior to the current strain relief operation is the same direction as the movement direction 550 of the RCM 522 from the position of the RCM 532(2) prior to the current strain relief operation to a position of the RCM 522(2) after the current strain relief operation. Therefore, the control module determines that the RCM 522 is inserted too deeply into the workspace 510. Further, the control module estimates the insertion depth correction as the distance moved from the position of the RCM 532(2) at the start of the strain relief operation to the position of the RCM 522(5) at the end of the strain relief operation divided by the angle difference in radians between a first direction and a second direction. The first direction is the movement direction 540 from the distal portion of the cannula 530(1) at the end of the previous strain relief operation to the distal portion of the cannula 520(1) at the start of the current strain relief operation. The second direction is the movement direction 550 of the RCM 522(2) during the current strain relief operation. In some examples, the control module generates an output detectable by a human operator that is representative of the insertion depth correction. The output can be a visible output, an audible output, a haptic output, a command to retract or insert a component, and/or the like.

As shown in FIG. 5D a cannula 520(3) is inserted into an opening 512(3) of a workspace 510(3) along an insertion axis 524(3). The pose of the cannula 520(3) reflects a pose after a prior joint float operation, such as a prior strain relief operation, a prior clutch operation, and/or the like. As further shown in FIG. 5D, cannula 520(3) is inserted too shallowly into the workspace because a RCM 522(3) associated with cannula 520(3) is located at the exterior of the workspace above an opening. As shown in FIG. 5E, the operator has generated commands to control an instrument (not shown) during a procedure. As a result of the commands, a repositionable structure (not shown) supporting cannula 520(4) has caused a change in the pose of cannula 520(4) from a first pose of the cannula 530(4) such that insertion axis 524(4) of the cannula 520(4) has rotated counterclockwise and the distal portion of the cannula 520(4) has moved in movement direction 560 to the right. As shown in FIG. 5F, the control module performs a strain relief operation. Lateral forces 562 on the cannula 520 cause the cannula to move to the left from a first pose of the cannula 530(5) prior to the strain relief operation to a second pose of the cannula 520(5) after the strain relief operation. The lateral forces 562 on the right side of the opening 512(5) of the workspace 510(5) against the cannula 520 results in movement from the first pose of the cannula 530(5) to the second pose of the cannula 520(5). As a result, the RCM 522 likewise moves in movement direction 570 from a position of the RCM 532(5) at the start of the strain relief operation to a position of the RCM 522(5) at the end of the strain relief operation.

The control module determines that the movement of the RCM 522(3) at the end of the prior joint float operation to the position of the RCM 522(4) at the start of the current strain relief operation is a direction from the movement of the RCM from the position of the RCM 532(5) at the start of the strain relief operation to the position of the RCM 522(5) at the end of the strain relief operation. Therefore, the control module determines that the RCM 522 is inserted too shallowly into the workspace 510. Further, the control module estimates the insertion depth correction as the distance moved from the position of the RCM 532(5) at the start of the strain relief operation to the position of the RCM 522(5) at the end of the strain relief operation divided by the angle in radians between the position of the RCM 522(3) after the prior joint float operation and the position of the RCM 522(4) prior to the current strain relief operation. In some examples, the control module generates an output detectable by a human operator that is representative of the insertion depth correction. The output can be a visible output, an audible output, a haptic output, a command to retract or insert a component, and/or the like.

In some examples, when supporting a cannula 286 or instrument 270 that is held to be coincident with an RCM 274 of the repositionable structure, the repositionable structure moves in concert with the RCM 274. In such examples, the motion of the RCM 274 is also the motion of the associated cannula 286 (or instrument 270), The control module can determine this motion of the RCM 274 by determining the motion of the relevant component(s) of the repositionable structure, of the RCM 274, of the relevant component(s) of the cannula 286 (or instrument 270), and/or the like.

At a process 308, the control module determines whether the placement quality is acceptable. The control module determines whether the placement quality of the component is acceptable based on the amount of motion of the RCM, such as the magnitude of motion of the RCM, the direction of motion of the RCM, and/or the speed of motion of the RCM, during the current strain relief operation. The control module can determine that the placement quality is of lower quality if the motion of the RCM exceeds an RCM distance threshold and/or an RCM speed threshold. The control module can determine that the placement quality is of higher quality in response to the motion of the RCM not exceeding the RCM distance threshold and/or the RCM speed threshold. For example, the control module can determine whether a distance between the position of the RCM at the start of the current strain relief operation stored during process 402 and the position of the RCM at the end of the current strain relief operation stored during process 410 exceeds an RCM distance threshold. When the placement quality is acceptable (e.g., the amount of motion of the RCM during the strain relief operation is not greater than the RCM distance threshold), then the method 300 proceeds to process 302, described above, to detect subsequent indications that a strain relief operation should be performed. When the placement quality is not acceptable (e.g., the amount of motion of the RCM during the strain relief operation is greater than the RCM distance threshold), then the method proceeds to process 310.

At process 310, the control module generates a warning that the placement of the component is outside of the target range. The warning can be a human-detectable indication comprising one or more visual indications, audio indications, haptic indications, and/or the like. In some examples, the warning can provide feedback indicating placement quality at any level of quantization, including a good placement/bad placement warning, a spectrum showing placement quality over a continuum, a spectrum showing placement depth over a continuum, a warning when placement quality is below a placement quality threshold, and/or the like. In some examples, the control module can estimate an insertion depth correction amount. In some examples, the control module generates an output detectable by a human operator that is representative of the insertion depth correction. The output can be a visible output, an audible output, a haptic output, a command to retract or insert a component, and/or the like. The control module can generate a warning that includes this estimated insertion depth correction amount, along with an indication as to whether the RCM 274 is inserted too deeply or too shallowly. These indications can aid the operating in correcting the placement of the RCM 274 to be at a depth that is within a target range. The control module approximates the insertion depth correction amount based on the amount of motion during the current strain relief operation. In some examples, the control module estimates the insertion depth correction amount as the distance moved during strain relief operation divided by the angular difference (in radians or degrees or any appropriate measurement system) between: (1) a first direction of motion of the distal portion of the cannula 286 from the end of the previous strain relief operation to the start of the current strain relief operation with (2) a second direction of motion of the RCM 274 during the current strain relief operation. As referred to herein, a joint float operation includes a prior strain relief operation, a clutch operation, and/or the like. In some examples, the control module can further determine the insertion depth based on the insertion angle of the cannula 286 and/or the instrument 270. In particular, if the insertion angle is small relative to an insertion angle threshold, then the angle of the cannula 286 and/or the instrument 270 can affect the insertion depth. The control module can determine the insertion angle threshold from the initial docking position, the type of procedure being performed, and/or the like.

Method 300 then repeats by proceeding back to process 302, described above, to detect subsequent indications that a strain relief operation should be performed.

Some examples of control systems, such as the control system 130 of FIG. 1 can include non-transient, tangible, machine-readable media that include executable code that when executed by a processor system (e.g., the processor system 140 of FIG. 1) can cause the processor system to perform the processes of method 300 and/or method 400. Some common forms of machine-readable media that can include the processes of method 300 and/or method 400 are, for example, floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, and/or any other medium from which a processor or computer is adapted to read.

Although illustrative embodiments have been shown and described, a wide range of modification, change and substitution is contemplated in the foregoing disclosure and in some instances, some features of the embodiments may be employed without a corresponding use of other features. One of ordinary skill in the art would recognize many variations, alternatives, and modifications. Thus, the scope of the invention should be limited only by the following claims, and it is appropriate that the claims be construed broadly and in a manner consistent with the scope of the embodiments disclosed herein.

1. A computer-assisted system comprising:
a repositionable structure configured to pivot a component about a remote center of motion (RCM) during operation, wherein the repositionable structure comprises a plurality of joints; and
a processor system communicatively coupled to the repositionable structure;
wherein the processor system is configured to:
command the repositionable structure to perform a strain relief operation by floating one or more joints of the plurality of joints, wherein floating the one or more joints facilitates external manipulation of the repositionable structure that moves the RCM,
determine a motion of the RCM occurring during the strain relief operation, and
determine a placement quality of the component based on the motion of the RCM.

2. The computer-assisted system of claim 1, wherein to perform the strain relief operation, the processor system is configured to end the strain relief operation in response to determining that a motion of the component during the strain relief operation:
has a distance greater than a component distance threshold; or
has moved the component to a position beyond the component distance threshold from a reference position; or
has a speed greater than a component speed threshold; or
is in a disallowed direction.

3-4. (canceled)

5. The computer-assisted system of claim 1, wherein to perform the strain relief operation, the processor system is configured to:
end the strain relief operation in response to determining that a motion of the component has stopped; or
end the strain relief operation in response to determining that a duration of time has elapsed since a start of the strain relief operation.

6. The computer-assisted system of claim 1, wherein to determine the motion of the RCM occurring during the strain relief operation, the processor system is configured to:
compare a first position of the RCM at an end of the strain relief operation with a second position of the RCM at a start of the strain relief operation; or
compare the first position of the RCM at the end of the strain relief operation with a third position of the RCM at an end of a previous strain relief operation.

7. The computer-assisted system of claim 1, wherein to determine the placement quality, the processor system is configured to:
determine the placement quality based on at least one motion characteristic selected from the group consisting of: a magnitude of the motion of the RCM, a direction of the motion of the RCM, and a speed of the motion of the RCM.

8. The computer-assisted system of claim 1, wherein to determine the placement quality, the processor system is configured to:
determine the placement quality is of lower quality in response to the motion of the RCM exceeding an RCM distance threshold or an RCM speed threshold, and as a better quality in response to the motion of the RCM not exceeding the RCM distance threshold or the RCM speed threshold.

9. The computer-assisted system of claim 1, wherein to determine the placement quality, the processor system is configured to compare:
a first direction of a net motion of a portion of the component from a position at an end of a previous strain relief operation to a position at a start of the strain relief operation, and
a second direction of the motion of the RCM during the strain relief operation.

10. (canceled)

11. The computer-assisted system of claim 9, wherein to determine the placement quality, the processor system is further configured to:
determine an angle between the first direction and the second direction; and
in response to determining that the angle is past an angle threshold determine that a depth of the RCM is shallower than a target depth range in response to the second direction being in a same direction as the first direction.

12. The computer-assisted system of claim 9, wherein to determine the placement quality, the processor system is further configured to:
determine an angle between the first direction and the second direction; and
in response to determining that the angle is past on angle threshold, determine that a depth of the RCM is deeper than a target depth range in response to the second direction being in an opposite direction to the first direction.

13-14. (canceled)

15. The computer-assisted system of claim 1, further comprising:
an input device configured to interact with a human operator;
wherein the processor system is further configured to:
in response to operator commands received at the input device while in a teleoperation mode, teleoperate the component by moving the repositionable structure.

16. The computer-assisted system of claim 1, wherein to command the repositionable structure to perform the strain relief operation, the processor system is configured to: initiate the strain relief operation in response to:
a defined period of time having passed since a previous strain relief operation; or
the component having stopped moving for a defined duration of time; or
the repositionable structure having stopped moving for the defined duration of time.

17-18. (canceled)

19. The computer-assisted system of claim 1, wherein the component comprises an instrument supported by the repositionable structure, the computer-assisted system further comprising:
an input device configured to interact with a human operator;
wherein the processor system is further configured to: while in a teleoperation mode, teleoperate the instrument by moving the repositionable structure in response to operator commands received at the input device; and
wherein to command the repositionable structure to perform the strain relief operation, the processor system is configured to:
initiate the strain relief operation in response to determining that:
the instrument is in view of an imaging device while the instrument is not being moved by teleoperation; or
the input device has moved by more than an input device distance threshold.

20-21. (canceled)

22. The computer-assisted system of claim 1, wherein the processor system is further configured to:
determine an insertion depth correction based on a distance of the motion of the RCM during the strain relief operation and an angle between a first position of the RCM after a prior joint release operation and a second position of the RCM prior to the strain relief operation; and
generate an output detectable by a human operator representative of the insertion depth correction.

23. (canceled)

24. A method comprising:
commanding, by a processor system, a repositionable structure to perform a strain relief operation by floating one or more joints of a plurality of joints of the repositionable structure, wherein floating the one or more joints facilitates external manipulation of the repositionable structure that moves a remote center of motion (RCM), wherein the repositionable structure is configured to pivot a component about the RCM during operation;
determining, by the processor system, a motion of the RCM occurring during the strain relief operation; and
determining, by the processor system, a placement quality of the component based on the motion of the RCM.

25. The method of claim 24, wherein performing the strain relief operation comprises ending the strain relief operation in response to determining that a motion of the component during the strain relief operation:
has a distance greater than a component distance threshold; or
has moved the component to a position beyond the component distance threshold from a reference position; or
has a speed greater than a component speed threshold; or
is in a disallowed direction.

26-27. (canceled)

28. The method of claim 24, wherein performing the strain relief operation comprises:
ending the strain relief operation in response to determining that a motion of the component has stopped; or
ending the strain relief operation in response to determining that a duration of time has elapsed since a start of the strain relief operation.

29-31. (canceled)

32. The method of claim 24, wherein determining the placement quality comprises:
comparing:
a first direction of a net motion of a portion of the component from a position at an end of a previous strain relief operation to a position at a start of the strain relief operation, and
a second direction of the motion of the RCM during the strain relief operation; and
in response to determining that a difference between the first direction and the second direction is past an alignment limit, determining that a depth of the RCM is outside a target depth range.

33-36. (canceled)

37. The method of claim 24, wherein the component comprises an instrument supported by the repositionable structure, and further comprising:
while in a teleoperation mode, teleoperating, by the processor system, the instrument by moving the repositionable structure in response to operator commands received at an input device configured to interact with a human operator,
wherein commanding the repositionable structure to perform the strain relief operation comprises:
initiating the strain relief operation in response to determining that the instrument is in view of an imaging device while the instrument is not being moved by teleoperation; or
initiating the strain relief operation in response to determining that the input device has moved by more than an input device distance threshold.

38-39. (canceled)

40. The method of claim 24, further comprising:
determining, by the processor system, an insertion depth correction based on a distance of the motion of the RCM during the strain relief operation and an angle between a first position of the RCM after a prior joint release operation and a second position of the RCM prior to the strain relief operation; and
generating, by the processor system, an output detectable by a human operator representative of the insertion depth correction.

41. (canceled)

42. One or more non-transitory machine-readable media comprising a plurality of machine-readable instructions which when executed by a processor system associated with a computer-assisted system are adapted to cause the processor system to perform a method comprising:
commanding a repositionable structure to perform a strain relief operation by floating one or more joints of a plurality of joints of the repositionable structure, wherein floating the one or more joints facilitates external manipulation of the repositionable structure that moves a remote center of motion (RCM), wherein the repositionable structure is configured to pivot a component about the RCM during operation;
determining a motion of the RCM occurring during the strain relief operation; and
determining a placement quality of the component based on the motion of the RCM.
